(19) United States
(12) Patent Application Publication
(10) Pub. No.: US 2024/0366856 A1
(43) Pub. Date: Nov. 7, 2024

(54) DEVICE FOR PREPARING AND DISPENSING ACTIVE INGREDIENT SOLUTIONS

(71) Applicant: Heraeus Medical GmbH, Wehrheim (DE)

(72) Inventors: Sebastian VOGT, Wehrheim (DE); Thomas KLUGE, Wehrheim (DE)

(21) Appl. No.: 18/649,253

(22) Filed: Apr. 29, 2024

(30) Foreign Application Priority Data
May 4, 2023 (EP) 23171450.2

(51) Int. Cl. A61M 3/02

(52) U.S. Cl. CPC A61M 3/0287; A61M 3/0237

(57) ABSTRACT

A device for producing an active ingredient solution, preferably an antiseptic wound-irrigating solution, having:
a connection element designed and configured for liquid-tight connection to a container;
a mandrel designed and configured for piercing a septum of a container;
a reservoir containing an active ingredient in solid form, and connectable via an outlet of the reservoir in a fluid-conducting manner to the connection element;
a liquid-permeable filter arranged at the outlet of the reservoir and configured to retain the active ingredient in solid form in the reservoir;
a fluidic element configured to receive a liquid from a container into the reservoir, configured to dispense a liquid from the reservoir;
wherein the device is configured to receive a liquid from a container into the reservoir, to dissolve the active ingredient in the liquid, and to dispense the dissolved active ingredient to a patient via the connection element.

### CROSS-REFERENCE TO RELATED APPLICATIONS

This application claims priority pursuant to 35 U.S.C. 119(a) to European Application No. 23171450.2, filed May 4, 2023, which application is incorporated herein by reference in its entirety.

### FIELD OF THE INVENTION

The present invention relates to the field of medical technology, in particular devices for preparing and dispensing active ingredient solutions—for example, wound-irrigating solutions.

### TECHNICAL BACKGROUND

Antiseptic active ingredients and antiseptic solutions have been known since the 19th century. An example of such active ingredients are hypochlorite salts and their solutions. The shelf-life of aqueous hypochlorite solutions is limited. The cause of this is mainly the slowly progressing conversion of the hypochlorite ions to chlorine ions. This means that the hypochlorite content of the solutions decreases with increasing storage time. In addition, a decomposition of

hypochlorite ions into chloride ions and oxygen is also possible.

Antiseptic hypochlorite solutions are known in the prior art, wherein various solutions have been proposed for improving storage stability.

Such solutions have been described, for example, in the following publications: U.S. Pat. No. 8,945,630B2, US2011/0236490A1, WO2021001789, US2019328776A1, WO2021134041A1, WO2022/038507A1, WO2020252433, WO2020174436A1, US243781, U.S. Pat. Nos. 3,749,672, 6,162,371, WO1991/003936, US641974, US2009/0258083, WO2010148004, WO2010148004, US2009/0148342A1, WO2016/100543A2, WO2020/089693A1, and WO2021/162736A1.

However, complete protection from the decomposition of the hypochlorite ions in aqueous solution remains challenging.

A further challenge is the provision of such solutions in sterile form, wherein the aim is that the containers are also to be sterile and stable. U.S. Pat. No. 4,964,261A and US2005232848A1, for example, address this task.

Solid precursors for producing hypochlorite-containing solutions are described in DE2712574A1 and U.S. Pat. No. 4,104,190A1.

### Preferred Embodiments

An object of the present invention is to solve one or more of the problems described above and further problems of the prior art. For example, the invention provides an active-ingredient-containing device with the aid of which a ready-to-use active ingredient solution can be produced quickly, easily, and economically using commercially available containers with sterile saline

solution or comparable solutions. Since the device according to the invention contains the active ingredient in solid form, the device can be stored for a longer period of time, even if it contains an active ingredient which is not permanently stable in aqueous solution. In addition, the sterilization of liquids is omitted during the production of the device according to the invention. Some active ingredients can be better sterilized in solid form, since they decompose more easily in solution
during sterilization. In some embodiments, the device according to the invention makes it possible to dispense the liquid produced therein directly to a patient—for example, using a nozzle which can be attached to the device according to the invention.

These objects are achieved by the methods and devices described herein, and in particular those which are described in the claims.

Preferred embodiments of the invention are described below.

A first embodiment relates to a device for producing an active ingredient solution, preferably an antiseptic wound-irrigating solution, having:
a connection element which is designed and configured for liquid-tight connection to a container;
a mandrel which is designed and configured for piercing a septum of a container;
a reservoir which contains an active ingredient in solid form, and can be connected via an outlet of the reservoir in a fluid-conducting manner to the connection element;
a liquid-permeable filter which is arranged at the outlet of the reservoir and is configured to retain the active ingredient in solid form in the reservoir;
a fluidic element which is configured to receive a liquid from a container into the reservoir, and/or is configured to dispense a liquid from the reservoir;
wherein the device is designed and configured to receive a liquid from a container into the reservoir, to dissolve the active ingredient in the liquid, and to dispense the dissolved active ingredient to a patient via the connection element.

A second embodiment relates to a device according to embodiment 1, wherein the mandrel has a hollow interior which is configured to receive a liquid from a container into the reservoir.

A third embodiment relates to a device according to one of the preceding embodiments, wherein the fluidic element has a plunger which is movably arranged within the reservoir and closes off the reservoir in a liquid-tight manner.

A fourth embodiment relates to a device according to one of the preceding embodiments which has a first component and a second component which can be detachably connected to one another.

A fifth embodiment relates to a device according to embodiment 4, wherein the first component contains the connection element and the mandrel, and/or wherein the second component contains the reservoir, the filter, and the fluidic element.

A sixth embodiment relates to a device according to embodiment 4 or 5, wherein the device also has a thread, by means of which the first component can be connected to the second component.

A seventh embodiment relates to a device according to one of the preceding embodiments, wherein the device is completely sterilized, and preferably is completely non-pyrogenic.

An eighth embodiment relates to a device according to one of the preceding embodiments, wherein the device also has a spray nozzle which is configured to dispense the active ingredient in dissolved form.

A ninth embodiment relates to a device according to embodiment 8, wherein the spray nozzle can be detachably connected in a fluid-conducting manner to the connection element.

A tenth embodiment relates to a device according to one of the preceding embodiments, wherein the active ingredient is an antiseptic which is preferably selected from the group consisting of calcium hypochlorite, magnesium hypochlorite, sodium hypochlorite, sodium percarbonate, sodium peroxide, calcium peroxide, urea peroxide, triisocyanuric chloride, sodium-N-chloro-4-methylbenzene sulfonamide, sodium-N-chlorobenzene sulfonamide, octenidine, polyhexanide, and chlorhexidine digluconate.

An eleventh embodiment relates to a device according to one of the preceding embodiments, wherein the connection element has a first feedthrough which is configured to receive a liquid from a container into the reservoir.

A twelfth embodiment relates to a device according to one of the preceding embodiments, wherein the connection element has a second feedthrough which is configured to dispense a liquid from a container, wherein the dispensing is preferably possible in a state in which the device is connected to the reservoir via the first feedthrough according to embodiment 11.

A thirteenth embodiment relates to a device according to one of the preceding embodiments, wherein the reservoir is permeable to gamma rays and/or electron beams.

A further aspect relates to a kit having a device according to one of embodiments 1 through 13 and a container having a medically compatible liquid, wherein the device can be detachably connected to the container, wherein the kit is packed together in a germ-tight manner, preferably

is completely sterilized, and more preferably is completely pyrogen-free.

Another aspect relates to an active ingredient for use in a medical treatment method, wherein the active ingredient is dissolved in a liquid, and subsequently administered in dissolved form, by means of a device according to one of the preceding embodiments immediately before

administration to a patient.

A further aspect relates to a method for preparing an active ingredient solution, which comprises the following steps:
a) receiving a liquid from a container using a device according to one of embodiments 1 through 13 into the reservoir of the device,
b) mixing the active ingredient within the reservoir with the liquid received in step a),
c) thereby obtaining an active ingredient solution,
d) optionally dispensing the active ingredient solution into the container.

Another aspect relates to the use of a device according to one of embodiments 1 through 13 for producing an active ingredient solution.

### BRIEF DESCRIPTION OF THE DRAWINGS

FIG. 1 shows a first part of a device which can be connected to a container.

FIG. 2 shows a container which can be connected to a device according to the invention.

FIG. 3 shows a first part of a device according to the invention which is attached to a container.

FIG. 4 shows a second part of a device according to the invention with a reservoir for an active ingredient and a fluidic element for transporting a liquid from a container.

FIG. 5 shows a device according to the invention which is connected to a container.

FIG. 6 shows a first part of a device according to the invention which has a thread for connection to a closure or to a second part of the device.

FIG. 7 shows a first part of a device according to the invention according to FIG. 6 which is connected via the thread to a second part of the device.

FIG. 8 shows a first part of a device according to the invention according to FIG. 6, which is connected to a nozzle via the thread.

FIG. 9 shows a device which is connected to a dispensing device.

FIG. 10 shows a device which is provided with a nozzle and a shield.

FIG. 11 shows a nozzle with a joint which can be used with a device according to the invention.

### DETAILED DESCRIPTION

With respect to the embodiments described herein, the elements of which “have,” “contain,” or “comprise” a particular feature (for example, a material), in principle, a further embodiment is always contemplated in which the relevant element consists solely of the feature, i.e., does not comprise any other constituents. The words, “comprise” or “comprising,” are used herein synonymously with the words, “contain,” “containing,” “have,” or “having.”

In one embodiment, if an element is denoted by the singular, an embodiment is also contemplated in which more than one such element is present. The use of a term for an element in the plural in principle also encompasses an embodiment in which only a single corresponding element is included. Unless otherwise indicated or clearly excluded from the context, it is possible in principle, and is hereby clearly contemplated, that features of different embodiments may also be present in the other embodiments described herein. Likewise, it is contemplated in principle

that all features described herein in connection with a method are also applicable to the products and devices described herein, and vice versa. All such considered combinations are not explicitly listed in all instances, simply in order to keep the description brief. Technical solutions known to be equivalent to the features described herein are also intended in principle to be encompassed by the scope of the invention.

A first embodiment describes a device for producing an active ingredient solution, having:
a connection element which is designed and configured for liquid-tight connection to a container;
a mandrel designed and configured for piercing a septum of a container;
a reservoir which contains an active ingredient in solid form, and can be connected via an outlet of the reservoir in a fluid-conducting manner to the connection element;
a liquid-permeable filter which is arranged at the outlet of the reservoir and is configured to retain the active ingredient in solid form in the reservoir;
a fluidic element which is configured to receive a liquid from a container into the reservoir, and/or is configured to dispense a liquid from the reservoir;
wherein the device is designed and configured to receive a liquid from a container into the reservoir, to dissolve the active ingredient in the liquid, and to dispense the dissolved active ingredient to a patient via the connection element.

The device comprises an active ingredient. The active ingredient is preferably an antiseptic active ingredient. The active ingredient solution is preferably an antiseptic wound-irrigating solution. The terms, “antiseptically” and “antimicrobially,” are used synonymously herein, and denote a bactericidal or fungicidal, i.e., germicidal, effect of an active ingredient. The device can furthermore contain an excipient. The active ingredient can be present as a mixture with such an

excipient. The excipient can comprise, for example, a buffer substance. In one embodiment, the excipient is selected from the group consisting of glucuronic acid, galacturonic acid, gluconic acid lactone, sodium gluconate, sodium hydrogen sulfate, sodium dihydrogen phosphate, calcium dihydrogen phosphate, citric acid, and monosodium citrate.

The device has a connection element. The connection element is designed and configured for fluid-tight connection of the device to a container. Such containers can, for example, be packages in which physiological saline solutions, infusion solutions, and similar liquid medical products are commercially available. For example, bottles or film bags are used for this purpose. These containers often consist of plastic. The container can be a container with a flexible,

preferably elastically deformable, outer wall, so that a liquid contained therein can be released from the container by compressing the container. The container can comprise a septum which can be pierced by the mandrel of the device.

The connection element of the device can preferably be connectable to a container in a form-fit. For example, the connection element can be attachable to the opening of a plastic bottle

or the connection of a filmpouch.

The device further contains a mandrel which is designed and configured for piercing a septum of a container. For this purpose, the mandrel can have a tapered shape, with the tip of which a septum of a container can be penetrated by manual exertion of pressure of the mandrel onto the septum. Comparable structures are used for connecting infusion sets to containers with infusion solutions.

The device furthermore has a reservoir. The reservoir contains an active ingredient in solid form—for example, as powder. The reservoir can have a substantially cylindrical shape, similar to the interior of a syringe.

The device can be configured to dispense the contained active ingredient into the container in dissolved form.

A filter is arranged at an outlet of the reservoir. This filter is configured to retain the active ingredient in the reservoir as long as the active ingredient is not dissolved in a liquid. In this way, it can be prevented that active ingredient be delivered to a patient in undissolved form.

### However,

it is desirable that the active ingredient only be able to be delivered to a patient in exclusively dissolved form. For this purpose, the filter can preferably be liquid-permeable. The filter is preferably gas-permeable. A gas-permeable filter makes it possible, for example, to fill the reservoir with active ingredient more easily, in particular during the production of the device according to the invention. For this purpose, the active ingredient can preferably be introduced into the reservoir using compressed air or a vacuum, wherein the gas-permeable filter allows the gas to pass through, but retains the active ingredient in the reservoir.

The filter can, for example, have the shape of a disk. The filter preferably has an open porosity. In some embodiments, the filter has open pores having a pore size less than 5 μm, preferably less than 3 μm, and particularly preferably less than 1 μm. This pore size can be determined by excluding spherical particles with a corresponding particle diameter. It is therefore a nominal pore size.

The filter can be fastened by a holder within the reservoir. The holder can be designed, for example, in the form of a clamping ring. Optionally, the filter can be fastened in a recess in a wall of the reservoir.

The filter can have, for example, polyethylene, polyurethane, polyimide, polyether sulfone, polyvinylidene fluoride, or polytetrafluoroethylene.

The filter can be a woven filter or a non-woven filter.

In some embodiments, the reservoir comprises a second filter which divides the reservoir into two separate regions, i.e., a first region and a second region. The second filter can be fastened within the reservoir by a second holder. The first region can contain a first active ingredient, and the second region can contain a second active ingredient which differs from the first active ingredient. The first active ingredient and the second active ingredient are preferably each in a solid form—for example, a powdered form. Such an embodiment enables the production

of an active ingredient solution with two different active ingredients, without the two active ingredients coming into contact with one another in solid form during the storage of the device.

The reservoir can be dimensioned such that the ratio of the volumes of the reservoir and the container is in a ratio of 1:25 to 1:500. The volume of the container can, for example, be 250 mL, 500 mL, or 1,000 mL. Accordingly, the volume of the reservoir can, for example, be 0.5 to 10 mL, 1 to 20 mL, or 2 to 40 mL.

The device also has a fluidic element. The fluidic element is preferably configured to receive a liquid from a container into the reservoir. Alternatively or additionally, the fluidic element

can be configured to dispense a liquid from the reservoir. The fluidic element is preferably configured to actively convey a liquid. The fluidic element can, for example, have a plunger for conveying a liquid. The fluidic element can have a syringe or a pump.

In one embodiment, the mandrel has a hollow interior which is configured to receive a liquid from a container into the reservoir. In this case, the mandrel can be designed, for example, in the form of a hollow needle. The mandrel can, for example, consist of plastic or comprise aplastic.

Preferably, the mandrel is sufficiently stiff to penetrate a septum of a commercially available container for physiological saline solution, but not so hard and solid that there is a risk of injury to medical personnel. In one embodiment, the mandrel is not a metal needle.

In one embodiment, the fluidic element has a plunger which is movably arranged within the reservoir and closes off the reservoir liquid-tight. The fluidic element can therefore be designed, for example, as a syringe or syringe pump. The fluidic element can also have other types of means for moving a liquid, e.g., a membrane pump, peristaltic pump, compressed air cartridge, or a connection for an external pressure or vacuum source.

In one embodiment, the device has a first component and a second component which can be detachably connected to one another. For example, the first component can contain the connection element and the mandrel, and/or the second component can contain the reservoir, the filter, and the fluidic element. The first component and the second component can be connectable to one another by a connecting element, preferably a form-fitting connection element
for example, by means of a thread or snap hook. A preferred thread is a luer lock thread. The first component can preferably have an external thread which can be connected to an internal thread of the second component. The second component can have an external thread which can be connected to an internal thread of the first component.

In one embodiment, the first component can be configured to be fluidically connected to a container, while the second component is disconnected from the first component and optionally

replaced by another device—for example, by a closure or a nozzle. The first component can therefore serve as a type of multifunctional quick connector for other components. The second component can comprise a syringe, particularly preferably a luer lock syringe.

As shown above, the device can comprise a connection element in order either to connect individual parts of the device according to the invention—in particular, the first component and the second component—to one another, or to connect the device to further devices, or both. In this case, either a single connection element can be configured to alternatively be connected to a plurality of different parts, or several connection elements can be provided at different positions of the device. A connection element is preferably a form-fitting connection element. An example

of such a connecting element is a thread. In some embodiments, the connection element is a luer lock thread.

In one embodiment, the device has a connection element, by means of which the first component can be connected to the second component. In some embodiments, the same connection element is additionally configured to connect the device to a closure, a nozzle, or another dispensing device. Alternatively, individual connecting elements that are different from one another can be provided for these purposes.

In one embodiment, the device is completely sterilized. In one embodiment, the device is completely pyrogen-free. The term, “sterilized,” refers to the device having been subjected to a method for removing or killing pathogens, so that the device can be used medically in accordance with the usual standards, such as, for example, the DIN EN 556-1 standard. Methods for sterilization comprise, for example, autoclaving, irradiation with electron beams or gamma rays, or treatment with disinfecting substances—for example, ethylene oxide. Similarly, the term, “non-

pyrogenic,” refers to the device having been subjected to a treatment customary in the art for removing or decomposing pyrogens. Whether the device is non-pyrogenic can be tested with the aid of the Limulus Amebocyte Lysate (LAL) test according to Ph. Eur. Chapter 2.6.14. The standards or documents that are current as of the priority date of the present application apply in each case.

In one embodiment, the device furthermore has a nozzle which is configured to dispense the active ingredient in dissolved form. The nozzle can have a substantially cylindrical hollow body tapering to a point at one end. The nozzle can optionally comprise a joint which allows the dispensing of a liquid in different directions by laterally bending an opening of the nozzle relative to the entire device.

The nozzle can preferably be connected in a detachable, fluid-conducting manner to the connection element. This can be made possible via a form-fitting connection element—for example, a thread, preferably a luer lock thread.

The device can furthermore comprise a shield. The shield can be configured to protect parts of the device from spatter of the discharged liquid, in particular an active ingredient solution. The shield can be arranged on, or in spatial proximity to, the nozzle. The shield can have the shape of a disk. The shield can have a concave curvature similar to a parabolic antenna. The

shield can have a centrally arranged recess which is configured to attach the shield to an output of the device.

In one embodiment, the active ingredient contained in the device is an antiseptic. In one embodiment, the antiseptic is selected from the group consisting of calcium hypochlorite, magnesium hypochlorite, sodium hypochlorite, sodium percarbonate, sodium peroxide, calcium peroxide, urea peroxide, triisocyanuric chloride, sodium-N-chloro-4-methylbenzene sulfonamide, sodium-N-chlorobenzene sulfonamide, octenidine, polyhexanide, and chlorhexidine digluconate. In one embodiment, the antiseptic is a hypochlorite salt.

In one embodiment, the active ingredient is present as a powder having an average particle size of less than 300 μm, preferably less than 200 μm, and particularly preferably less than 100 μm. Due to the small particle size, rapid dissolution of the particles of active ingredient can be achieved. The particle size of the active ingredient can preferably lie above the exclusion volume of the filter. This mean diameter of the particles can be determined by fractional screening, as is customary in the art. In one embodiment, the active ingredient is present as a cylindrical pressed blank or as a cylindrical solidified melt.

In one embodiment, the connection element has a first feedthrough which is configured to receive a liquid from a container into the reservoir. The first feedthrough preferably forms a fluid-conducting connection between the connection element—in particular, the mandrel—and the reservoir.

In one embodiment, the device is configured for dispensing a liquid from a container. In this case, the dispensing is preferably possible in a state in which the device is fluidically connected to the reservoir via the first feedthrough described above. For example, with the aid of such an embodiment, liquid from a container connected to the device can first be received into the reservoir of the device, the active ingredient contained in the liquid can be dissolved, and the dissolved active ingredient can subsequently be dispensed into the container while the device is

connected to the container. The dissolved active ingredient can then be dispensed from the container to a patient via the device, while the device, in particular the first component of the device, is still connected to the container. If necessary, the device can be connected to a nozzle or another dispensing device for dispensing the released active ingredient, wherein the second component of the device can optionally be replaced with the nozzle or other dispensing device.

The liquid is preferably a medically compatible liquid. The liquid is preferably suitable for internal application.

In one embodiment, the reservoir is permeable to gamma rays and/or electron beams. This makes it possible to sterilize the interior and the contained active ingredient of the device using gamma rays and/or electron beams in order to obtain a completely sterilized device.

In one embodiment, the mandrel and the reservoir jointly define a transfer axis, wherein a dispensing of the liquid from the reservoir and/or into the reservoir can take place along this transfer axis. In one embodiment, the container and the reservoir jointly define a transfer axis, wherein the liquid can be dispensed from the reservoir and/or received into the reservoir can along this transfer axis.

Another aspect relates to a kit having a device according to one of the preceding embodiments and a container having a medically compatible liquid, wherein the device can be detachably connected to the container. In one embodiment, the components of the kit, viz., the device and the container, are packed together germ-tight. In one embodiment, the components of the kit are each fully sterilized. In one embodiment, the components of the kit are each completely pyrogen-free. The kit can furthermore contain a dispensing device for dispensing a liquid, e.g., a nozzle or rinsing device, e.g., a rinsing device according to EP4035605A1, which is hereby completely incorporated by way of reference. The medically compatible liquid can comprise an infusion solution or medical rinsing solution. The medically compatible liquid can comprise a physiological saline solution, a ringer solution, or a comparable salt-containing solution. The medically compatible liquid is preferably sterile and/or pyrogen-free.

A further aspect relates to an active ingredient for use in a medical treatment method, wherein the active ingredient is dissolved in a liquid immediately before administration to a patient and is subsequently administered in dissolved form. The active ingredient can be a substance which has a pharmacological effect. The active ingredient can have an antimicrobial effect, for example.

Another aspect relates to a medical treatment method, wherein the active agent is dissolved in a liquid using a device described herein immediately prior to administration to a patient as described herein and subsequently administered in dissolved form. The liquid is preferably a medically compatible liquid as described herein. The treatment method can, for example, comprise the disinfection of a fabric. The treatment method can comprise a surgical joint operation—for example, the implantation or the replacement of a joint implant. The joint implant can be a spacer, for example. The joint operation can comprise, for example, a surgical hip, knee, shoulder, or back operation. The treatment method can further comprise the therapy for an inflammatory state. The treatment method can, for example, comprise the treatment of a joint arthrosis or a joint infection.

A further aspect relates to a method for preparing an active ingredient solution, which comprises the following steps:
a) receiving a liquid from a container using a device described herein into the reservoir of the device,
b) mixing the active ingredient within the reservoir of the device with the liquid received in step a),
c) thereby obtaining an active ingredient solution,
d) optionally dispensing the active ingredient solution into the container.

The method can preferably be carried out in the sequence a), b), c) or a), b), c), d). The method can furthermore comprise a step in which the active ingredient solution is discharged from

the device or from the container. The active ingredient solution can preferably be discharged from the container via the device, as described in more detail elsewhere herein.

A further aspect relates to an active ingredient solution which can be produced according to a method shown above.

A further aspect relates to a use of a device described herein or a kit described herein for producing an active ingredient solution.

### Examples

The invention is further illustrated below using examples, which are, however, not to be understood as limiting. It will be apparent to a person skilled in the art that other equivalent means may be used similarly in place of the features described here.

### FIGURES

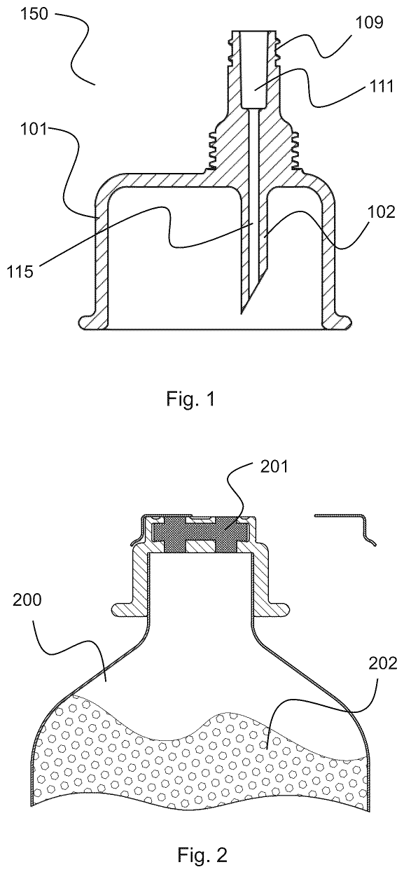

FIG. 1 shows by way of example a first component 150 of a device 100, which component can be connected to a container200. The first component150 of the device has a connection element101 which can be connected to a container in a form-fit—for example, with a connection provided for this purpose on the head of an infusion bottle or of a film pouch. The device also has a mandrel102 with which a septum201 of a container can be pierced. The mandrel102 has an inner cavity115 for guiding a liquid from the container into the device. The inner cavity is connected to a feedthrough111 of the device. The first component150 of the device100 also has a connecting element 109 with which the first component 150 of the device can be connected to a second component160 of the device. The connection element109 in this example is designed as a luer lock thread.

FIG. 2 shows a container200 which can be connected to a device according to the invention. The container200 contains a medically compatible liquid202 and has a septum201 which seals the container to the outside. The septum201 is formed from an elastic material—for example, silicone. The septum can additionally be protected by a seal which can be removed before the connection to the device according to the invention.

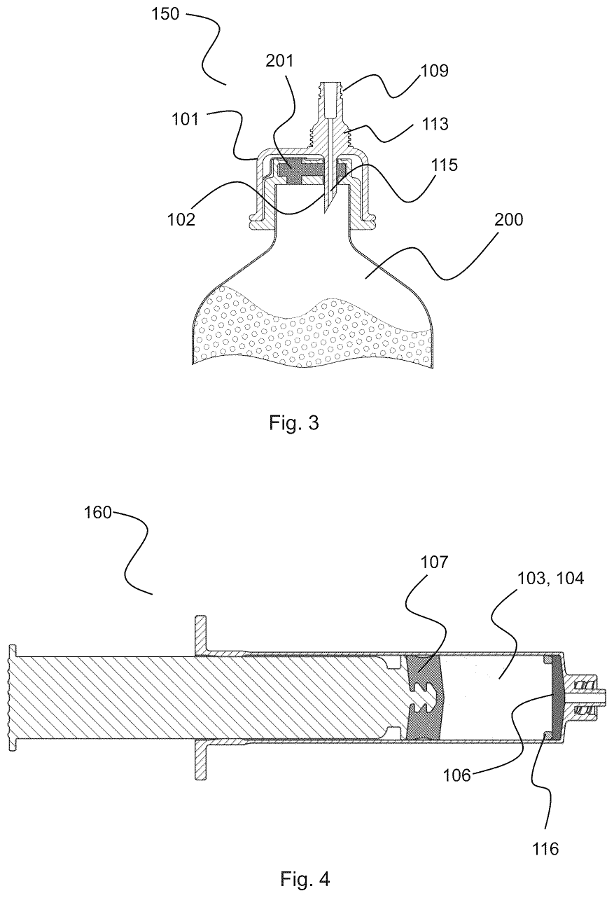

FIG. 3 shows a first component 150 of a device according to the invention which is attached to a container200. The mandrel102 penetrates the septum201 of the container200 so that the liquid202 can be transferred from the container through the cavity115 and the first feedthrough

109 into the device. The device comprises a first thread109 and a second thread113. Both threads (109, 113) of the first component150 are designed here as external threads.

FIG. 4 shows a second part 160 of a device according to the invention 100 with a reservoir 103 for an active ingredient 104 and a fluidic element 107 for transporting a liquid 202 from a container200. The fluidic element107 is designed here as a syringe. The active ingredient104 is arranged as a solid powder in the reservoir 103. The device also has a filter 106 for holding the active ingredient104 in the reservoir103. The active ingredient104 can pass through the filter106 only in dissolved form. The filter106 is fastened in the reservoir103 by a holder116 to seal the reservoir103 to the outside so that no solid active ingredient104 can pass through the filter 106 out of the reservoir 103. The second component 160 has a connection element for connection to the first component150. This connection element of the second component160 is complementary to the connecting element of the first component 150. In the example shown here, the second component160 has a luer lock internal thread.

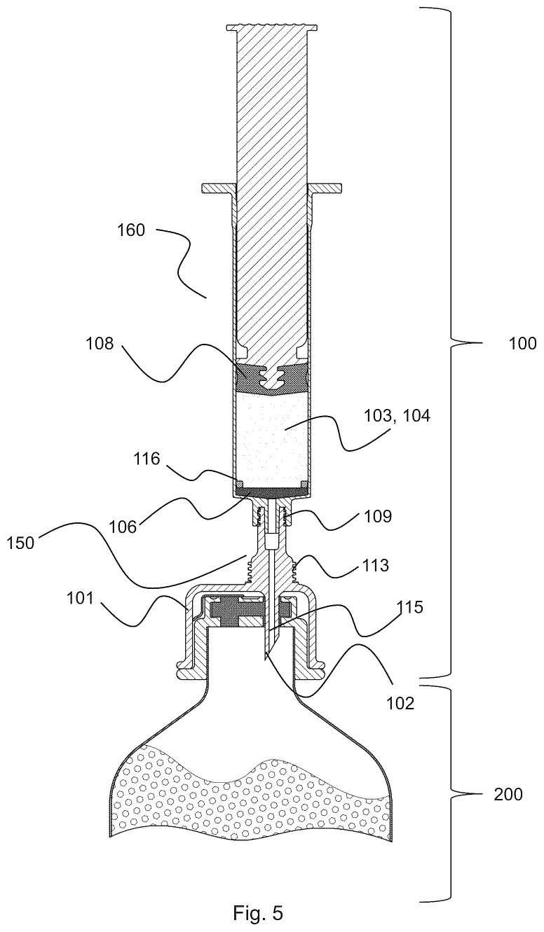

FIG. 5 shows a device according to the invention 100 which is connected to a container 200. The first component 1500 of the device is connected via a connecting element109 in a fluid-conducting and liquid-tight manner to the second component160 of the device. The connection element101 is connected to a container200, wherein the mandrel102 it is inserted through a septum of the container into the container. The interior of the container is connected to the reservoir103 through a cavity115 of the mandrel in a fluid-conducting manner. With the aid of a fluidic element, which has a plunger108 in this case, a liquid can now be fed from the container200 into the reservoir103.

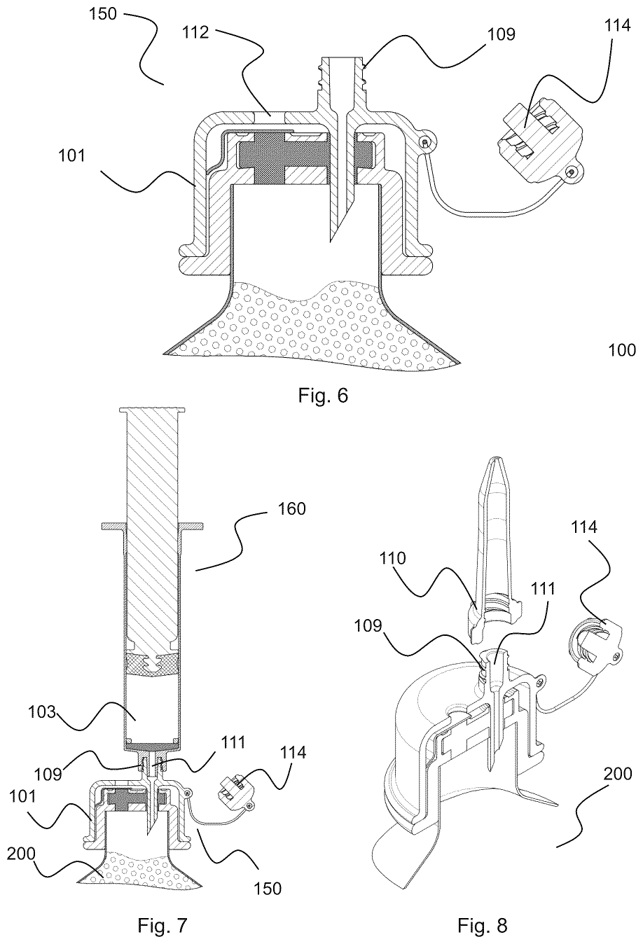

FIG. 6 shows a first component 150 of a device according to the invention which has a connecting element 109 for connection to a closure114 or to a second component of the device 160. In the embodiment shown here, the connecting element109 designed as a thread can alternatively be connected to different elements. The thread109 can either be connected to the second component of the device160 in a fluid-conducting manner, or can be closed in a fluid-tight manner using a closure114.

FIG. 7 shows a first component150 of a device according to the invention according to FIG. 6, which is connected to a second component160 of the device by the thread109. In this configuration, liquid can be removed from the container200 by the interaction of the two components150and160 of the device. The liquid can be pumped from the container200 via a first feedthrough111 into the reservoir103. Within the reservoir103, the active ingredient104 contained in the reservoir 103 can be dissolved with the aid of the liquid, and the active ingredient solution obtained in this way can be guided through the filter106 into the container200. Thereafter, the second component160 of the device can be removed, and the active ingredient solution can be dispensed from the container by means of the first component150 of the device
for example, dispensed to a patient.

FIG. 8shows a first component150 of a device according to the invention according to FIG. 6 which can be connected via a thread109 to a nozzle110. Using the nozzle110 and the feedthrough 111, the active ingredient solution produced using the device can be dispensed from

the container200. If necessary, the feedthrough111 can be sealed liquid-tight by attaching a closure114 to the connecting element109.

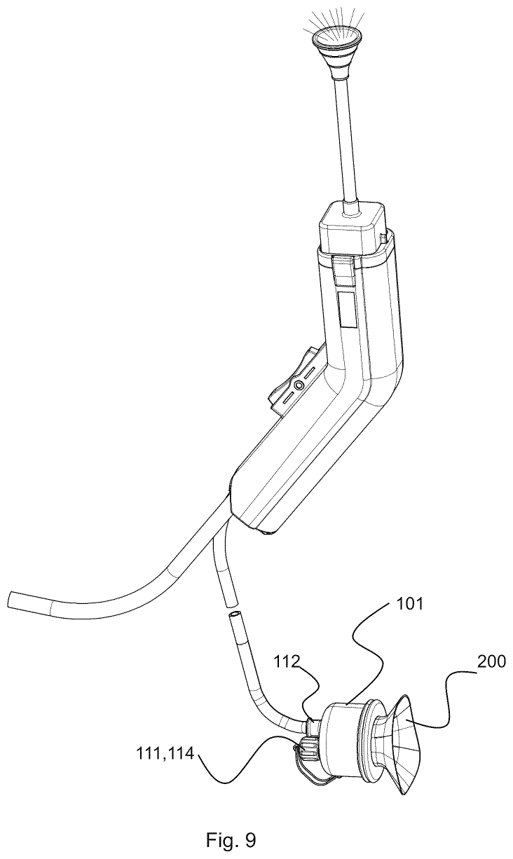

FIG. 9 shows a device according to the invention which is simultaneously connected to a container200 and to an external dispensing device. For this purpose, the device, in addition to a first feedthrough111 which is closed here liquid-tight by a closure114, comprises a second feedthrough112 which is configured for connection to an external dispensing device. In the configuration shown here, the first component150 is connected in a fluid-conducting manner to a container200 by means of the connecting element101 so that an active ingredient solution produced with the aid of the device can be dispensed from the container 200 via the second feedthrough 112.

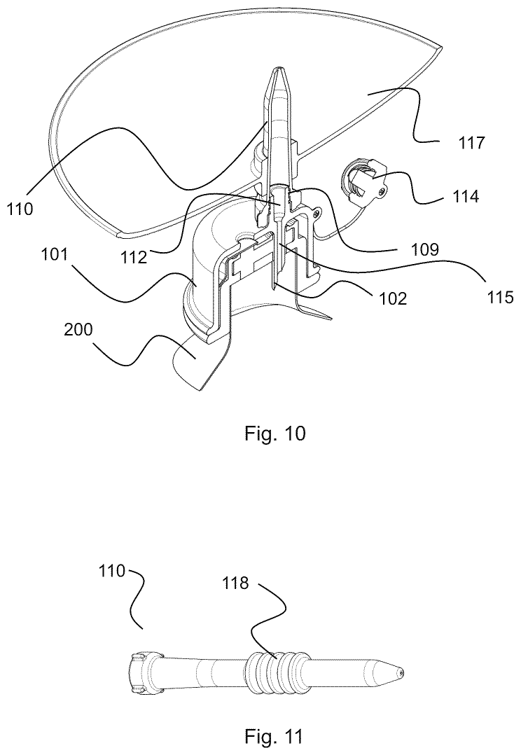

FIG. 10 shows a first component of a device which is provided with a nozzle110 and a shield117. The first component is configured to dispense an active ingredient solution from the container 200. The shield 117 in the example shown here has a central, cylindrical recess for fastening to the first component 150 and/or the nozzle 110. The shield can serve as a splash guard if an active ingredient solution is discharged by the nozzle110.

FIG. 11 shows a nozzle 110 with a joint 118 which can be used with a device according to the invention. With the aid of the joint118, the nozzle110 can be bent so that the outlet opening of the nozzle forms an angle with the connection of the nozzle that differs from 180°. An active ingredient solution can therefore be discharged in different directions without rotating the entire device.

### LIST OF REFERENCE SIGNS

100 Device
101 Connection element
102 Mandrel
103 Reservoir
104 Active ingredient
105 Outlet of the reservoir
106 Filter
107 Fluidic element
108 Plunger
109 First connecting element (for connecting the components)
110 Nozzle
111 First feedthrough (for receiving from the container)
112 Second feedthrough (for connection to pump or the like)
113 Second connection element (for nozzle)
114 Closure
115 Cavity of the mandrel
116 Filter holder
117 Shield
118 Joint
150 First component
160 Second component
200 Container
201 Septum
202 Liquid

1. A device (100) for producing an active ingredient solution, preferably an antiseptic wound-irrigating solution, having:
a connection element (101) which is designed and configured for liquid-tight connection to a container (200);
a mandrel (102) designed and configured for piercing a septum (201) of a container;
a reservoir (103) which contains an active ingredient (104) in solid form, and can be connected via an outlet (105) of the reservoir in a fluid-conducting manner to the connection
element (101);
a liquid-permeable filter (106) which is arranged at the outlet (105) of the reservoir, and is configured to retain the active ingredient (104) in solid form in the reservoir (103);
a fluidic element (107) which is configured to receive a liquid (202) from a container (200) into the reservoir (103), and/or is configured to dispense a liquid (202) from the reservoir (103);
wherein the device (100) is designed and configured to receive a liquid (202) from a container (200) into the reservoir, to dissolve the active ingredient (104) in the liquid (202), and to dispense the dissolved active ingredient to a patient via the connection element(101).

2. The device according to claim 1, wherein the mandrel (102) has a hollow interior (115) which is configured to receive a liquid (202) from a container (200) into the reservoir (103).

3. The device according to claim 1, wherein the fluidic element (107) has a plunger (108) which is movably arranged within the reservoir (103) and closes off the reservoir in a liquid-tight manner.

4. The device according to claim 1, which has a first component (150) and a second component (160) which can be detachably connected to one another.

5. The device according to claim 4, wherein the first component (150) contains the connection element (101) and the mandrel (102), and/or wherein the second component (160) contains the reservoir (103), the filter (106), and the fluidic element (107).

6. The device according to claim 4, wherein the device also has a connection element (109) by means of which the first component (150) can be connected to the second component(160).

7. The device according to claim 1, wherein the device is completely sterilized, and preferably is completely non-pyrogenic.

8. The device according to claim 1, wherein the device also has a nozzle (110) which is configured to dispense the active ingredient in dissolved form.

9. The device according to claim 8, wherein the nozzle (110) can be detachably connected in a fluid-conducting manner to the connection element (101).

10. The device according to claim 1, wherein the active ingredient is an antiseptic, which is preferably selected from the group consisting of calcium hypochlorite, magnesium hypochlorite, sodium hypochlorite, sodium percarbonate, sodium peroxide, calcium peroxide, urea peroxide, triisocyanuric chloride, sodium-N-chloro-4-methylbenzene sulfonamide, sodium-N-chlorobenzene sulfonamide, octenidine, polyhexanide, and chlorhexidine digluconate.

11. The device according to claim 1, wherein the connection element (101) has a first feedthrough (111) which is configured to receive a liquid (202) from a container (200) into the reservoir (103).

12. The device according to claim 1, wherein the connection element (101) has a second feedthrough (112) which is configured to dispense a liquid (202) from a container (200), wherein the dispensing is preferably possible in a state in which the device (100) is connected to the reservoir (103) via the first feedthrough (111) according to claim 11.

13. The device according to claim 1, wherein the reservoir (103) is permeable to gamma rays and/or electron beams.

14. The device according to claim 1, wherein the reservoir (103) furthermore has a second filter which divides the reservoir (103) into a first region and a second region.

15. A kit, having a device (100) according to claim 1 and a container (200) having a medically compatible liquid, wherein the device (100) can be detachably connected to the container (200), wherein the kit is packed together in a germ-tight manner, preferably is completely sterilized, and more preferably is completely non-pyrogenic.

16. An active ingredient for use in a medical treatment method, wherein the active ingredient is dissolved in a liquid and subsequently administered in dissolved form by means of a device according to claim 1 immediately before administration to a patient.

17. A method for producing an active ingredient solution, which comprises the following steps:
a) receiving a liquid (202) from a container (200) using a device (100) according to claim 1 into the reservoir (103) of the device,
b) mixing the active ingredient (104) within the reservoir (103) with the liquid (202) received in step a),
c) thereby obtaining an active ingredient solution,
d) optionally dispensing the active ingredient solution into the container (200).

18. A use of a device (100) according to claim 1 or a kit for producing an active ingredient solution.
